(19) United States
(12) Patent Application Publication
(10) Pub. No.: US 2024/0398331 A1
(43) Pub. Date: Dec. 5, 2024

(54) MEASURING THE HUMAN SKIN AGE THROUGH NEAR-INFRARED SPECTROSCOPY

(71) Applicant: TRINAMIX GMBH, Ludwigshafen Am Rhein (DE)

(72) Inventors: Michael HANKE, Ludwigshafen am Rhein (DE); Robert LOVRINCIC, Ludwigshafen am Rhein (DE); Florian PROELL, Ludwigshafen am Rhein (DE)

(21) Appl. No.: 18/697,235

(22) PCT Filed: Oct. 31, 2022
(86) PCT No.: PCT/EP22/80334

§ 371 Date: Mar. 29, 2024

(30) Foreign Application Priority Data
Nov. 2, 2021 (EP) 21205984.4

(51) Int. Cl. A61B 5/00

(52) U.S. Cl. CPC A61B 5/441; A61B 5/0075; A61B 5/7239; A61B 5/725; A61B 5/7267

(57) ABSTRACT

Disclosed herein is a computer-implemented method of optically determining an estimated age of a skin of a living being. The method includes:
i. using at least one sample reflection spectrum of at least one portion of the skin of the living being over a spectral measurement range, the spectral measurement range including at least one portion of the wavelength range of from 1 μm to 2.5 μm; and
ii. determining the estimated age of the skin of the living being by applying, to the sample reflection spectrum, at least one trained trainable model.

The trainable model is trained on a training dataset including a plurality of labeled reference reflection spectra. Each of the reference reflection spectra is acquired over a spectral range at least partially overlapping with the spectral measurement range of the sample reflection spectrum of step i.

### TECHNICAL FIELD

The invention relates to a method of optically determining an estimated age of a skin of a living being, a method of providing at least one recommendation to a human being, a training method of training a trainable model and a system for optically determining an estimated age of a skin of a living being. Generally, such methods and systems may be employed for various applications. Specifically, the methods and systems may for example be employed within the cosmetics sector. More specifically, the methods and systems may for example assist skin care. However, further kinds of applications are also possible.

### BACKGROUND ART

Due to the skin's complex structure and the interplay between its constituents with varying environmental conditions, e.g. temperature, humidity or stress level, obtaining objective quantitative measures for the general condition of the skin is a hard task. There are several such measures in use, among which are, for example, the hydration level through capacitive measurements, the sebum/lipid content using photometric methods, or the transepidermal water loss, which refers to a measure for the skin barrier functionality. All of them have in common that they target the uppermost skin layer, i.e. a few ten micrometers also called stratum corneum, and are thus subject to severe environmental and diurnal changes. Hence, in order to make meaningful statements about an objective state of the skin, measurements under controlled environmental conditions, e.g. provided by laboratory conditions at specialized dermatological institutions, are of paramount importance. Alternative approaches to determine skin conditions are based solely on advanced image processing and analysis in the visible wavelength range, e.g. RGB camera based. As an example, Skin Age Scan™ by Biotherm uses photographs taken with a smartphone in combination with general information about a user, e.g. an age or a skin type, for predicting a skin age. Such methods lack information on a molecular level and also suffer from many external interferences.

U.S. Pat. No. 6,501,982 B1 describes a noninvasive instrumentation and procedures for estimating the apparent age of human and animal subjects based on the irradiation of skin tissue with near-infrared light. The method of age estimation provides additional information about primary sources of systematic tissue variability due to chronological factors and environmental exposure. Therefore, categorization of subjects on the basis of the estimated apparent age is suitable for further spectral analysis and the measurement of biological and chemical compounds, such as blood analytes. Furthermore, age determination of subjects has particular benefit in assessment of therapies used to reduce the effects of ageing in tissue and measurement of tissue damage.

WO 2019/225870 A1 describes an unmanned apparatus for analyzing the skin of a face and the back of a hand. According to an embodiment the apparatus comprises: a kiosk including a touch screen; an opening positioned on the kiosk and allowing a hand of a user to be input thereinto; a first skin measurement module positioned on the upper end of the kiosk and including a first lighting module for illuminating the face of the user and a first camera module for photographing the face of the user; a second skin measurement module positioned on the opening and including a second lighting module for illuminating the hand of the user and a second camera module for photographing the hand of the user; and a control module for calculating a skin diagnosis result of the user on the basis of at least one of a first image photographed by the first skin measurement module and a second image photographed by the second skin measurement module.

Seker H et al. “Prediction of skin ages by means of multi-spectral light sources”, 2014 36th Annual International Conference of the IEEE Engineering in Medicine and Biology Society, IEEE, 26 Aug. 2014, pages 6736-6739, XP032675645, DOI: 10.1109/EMBC.2014.6945174 describes low-cost skin aging assessment apparatus by using light back-scatter intensity level of Red, Blue, Green and Infrared bands. This is further enhanced by using a machine learning method to accurately predict actual skin age. The proposed method appears to be highly capable of capturing multi-layer cellular changes exhibited by the biological skin aging process and predicting skin ages with a root-mean-square error of as low as 0.160 by using only four features based on the four multi-spectral light sources.

Overall, it is very difficult to define meaningful precise and reliable quantitative parameters for skin quality that can be the basis of targeted skin care steps, e.g. an application of different kinds of cosmetic products or medical procedures.

### Problem to be Solved

It is therefore desirable to provide methods and devices which are suited to provide precise and reliable parameters for skin quality.

### SUMMARY

This problem is addressed by a method of optically determining an estimated age of a skin of a living being, a method of providing at least one recommendation to a human being, a training method of training a trainable model and a system for optically determining an estimated age of a skin of a living being with the features of the independent claims. Advantageous embodiments which might be realized in an isolated fashion or in any arbitrary combinations are listed in the dependent claims as well as throughout the specification.

In a first aspect of the present invention, a computer-implemented method of optically determining an estimated age of a skin of a living being, specifically of a human being, is disclosed.

The term “computer” as used herein is a broad term and is to be given its ordinary and customary meaning to a person of ordinary skill in the art and is not to be limited to a special or customized meaning. The term specifically may refer, without limitation, to a device or to a combination or network of devices having at least one data processing means such as at least one processing unit. The computer, additionally, may comprise one or more further components, such as at least one of a data storage device, an electronic interface or a human-machine interface. The term “computer-implemented method” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one data processing means which is configured for performing at least one of the method steps of at least one of the methods according to the present invention. Specifically, each one of the method steps may be performed by using the computer and/or computer network. The method steps may at least partially be performed automatically, specifically without user interaction.

The term “optical” including any grammatical variations thereof as used herein is a broad term and is to be given its ordinary and customary meaning to a person of ordinary skill in the art and is not to be limited to a special or customized meaning. The term specifically may refer, without limitation, to an arbitrary physical property related to electromagnetical radiation in at least one of the visible, the ultraviolet and the infrared spectral range, specifically to infrared radiation. The infrared radiation may specifically comprise electromagnetic radiation in the range of 760 nm to 1000 μm, wherein the range of 760 nm to 1.5 μm is usually denominated as “near infrared spectral range” (NIR) while the range from 1.5 p to 15 μm is denoted as “mid infrared spectral range” (MidIR) and the range from 15 μm to 1000 μm as “far infrared spectral range” (FIR). Preferably, the optical radiation used for the typical purposes of the present invention is optical radiation in the infrared (IR) spectral range, more preferred, in the near infrared (NIR) and the mid infrared spectral range (MidIR), specifically the optical radiation having a wavelength of 1 μm to 2.5 μm. Further, the term optical may specifically relate to at least one spectral property of at least one measurement object, wherein the measurement object may specifically be or comprise at least a portion of at least one living being, more specifically at least a portion of a skin of the living being. The spectral property may specifically comprise at least one of an absorbance of the measurement object, an emissivity of the measurement object, a reflexivity of the measurement object and a transmissivity of the measurement object. The term “spectral” including any grammatical variations thereof, specifically “spectrum”, as used herein is a broad term and is to be given its ordinary and customary meaning to a person of ordinary skill in the art and is not to be limited to a special or customized meaning. The term specifically may refer, without limitation, to a partition of the optical wavelength range. A spectrum may be constituted by at least one optical signal defined by a signal wavelength and a corresponding signal intensity. Specifically, the spectrum may comprise spectral information relating to the measurement object, e.g. to a type and/or a composition of at least one material forming the measurement object.

The term “living being” as used herein is a broad term and is to be given its ordinary and customary meaning to a person of ordinary skill in the art and is not to be limited to a special or customized meaning. The term specifically may refer, without limitation, to an arbitrary organic organism, specifically a multicellular organism having a metabolism. Specifically, the living being may be or may comprise at least a portion of at least one human being. More specifically, the living being may be a human being. However, further options may be feasible. As an example, the living being may be or may comprise at least one portion of at least one animal. The living being may comprise a body, wherein the body may specifically refer to a biological body. Specifically, the living being may comprise at least one tissue, more specifically at least one organ. The living being may specifically have a skin. The living being may be in a living state or in a dead state.

The term “skin” as used herein is a broad term and is to be given its ordinary and customary meaning to a person of ordinary skill in the art and is not to be limited to a special or customized meaning. The term specifically may refer, without limitation, to an organ enveloping and/or covering a living being. Thus, the skin may be or may comprise at least one of an envelope organ; a body cover, specifically an outer body cover; a body shell, specifically an outer body shell. The skin may comprise a plurality of layers. At least one of the layers may comprise a plurality of sublayers. Specifically, from the outside to the inside, the skin may comprise at least one of an epidermis, a dermis and a subcutis, wherein, from the outside to the inside, the epidermis may comprise a stratum cornum, a stratum ludicum, a stratum granulosum, a stratum spinosum and a stratum basale. The skin may be configured for at least one of protecting the living being from environmental influences, maintaining a homeostasis of the living being and facilitating a metabolism of the living being. As an example, the skin may protect the human being from ultraviolet radiation or may facilitate a thermoregulation of the living being. In principle, the skin may comprise an arbitrary texture. The skin may be closed. The skin may comprise at least one of at least one opening, at least one pore and at least one protrusion. Specifically, the skin may comprise at least one of at least one hair or feather, at least one papillae and at least one gland, specifically at least one exocrine gland. As an example, the skin may comprise a plurality of sweat glands facilitating the thermoregulation. The skin may further comprise at least one receptor, e.g. a thermoreceptor. The texture of the skin or also environmental influences may specifically have an impact on a skin flora residing on the skin, wherein the skin flora may comprise a variety of microorganisms such as bacteria.

The term “estimated age of a skin” as used herein is a broad term and is to be given its ordinary and customary meaning to a person of ordinary skill in the art and is not to be limited to a special or customized meaning. The term specifically may refer, without limitation, to an age or age value or age bracket assigned to the skin based on at least one measurable physical property of the skin, specifically based on at least one spectral property of the skin. Thus, the estimated age of the skin may be an age or age value determined on the basis of the at least one measurable physical property, whereas the “real age” or “know age” is the age of the skin determined by the known age or date of birth of the living being.

The estimated age of the skin may deviate from the real or known age of the skin. Thus, as indicated, the skin may specifically be subject to a variety of different influences, both intrinsic and/or extrinsic, such as ultraviolet radiation, sweat or bacteria, which can be damaging for the skin. Firstly, intrinsic skin ageing, though genetically determined and inalterable, is not constant across different populations or even different anatomical sites on the same individual plus there is a number of potential components of extrinsic skin ageing, including nutrition, tobacco use and exposure to solar rays, resulting in a wide range of visible signs of aged skin even within genetically similar individuals of the same age, as it is e.g. known from Farage, M. A, et al. “Intrinsic and extrinsic factors in skin ageing: a review.” International Journal of Cosmetic Science 30.2 (2008): 87-95. Skin aging scores such as “SCINEXA” comprising 5 items indicative of intrinsic and 18 items highly characteristic of extrinsic skin ageing can help differentiating between intrinsic and extrinsic skin aging, as it is e.g. known from Vierkötter, Andrea, et al. “The SCINEXA: a novel, validated score to simultaneously assess and differentiate between intrinsic and extrinsic skin ageing.” Journal of dermatological science 53.3 (2009): 207-211. Chronic poor sleep quality is associated with increased signs of intrinsic ageing, diminished skin barrier function and lower satisfaction with appearance, as it is e.g. known from Oyetakin-White, P, et al. “Does poor sleep quality affect skin ageing?.” Clinical and experimental dermatology 40.1 (2015): 17-22. Further, daily diet can influence skin aging, as can be determined by measuring hydration, surface lipids and elasticity of the skin, as it is e.g. known from Nagata, Chisato, et al. “Association of dietary fat, vegetables and antioxidant micronutrients with skin ageing in Japanese women.” British Journal of Nutrition 103.10 (2010): 1493-1498. Lastly, extrinsic skin aging can also be caused by psychological stress, as it is e.g. known from Lee, C. M, R. E. B. Watson, and C. E. Kleyn. “The impact of perceived stress on skin ageing.” Journal of the European Academy of Dermatology and Venereology 34.1 (2020): 54-58. Contrarily, other influences such as skin care products or a healthy diet may have a positive impact on the skin. Thus, overall, the estimated age of the skin may deviate from the age of the living being comprising the skin. As an example, the estimated age of the skin may be higher than the age of the living being comprising the skin in case the skin was mainly subject to negative influences. Conversely, the estimated age of the skin may be lower than the age of the living being comprising the skin in case the skin was mainly subject to positive influences. Thus, the estimated age of the skin may generally depend on a physical state and/or a state of health of the skin. Of course, the state of health of the skin may again depend on an overall state of health of the living being and on an age of the living being.

As indicated, the estimated age of the skin of the living being is determined optically. Specifically, the estimated age of the skin may be determined by using at least one spectral property of the skin. In principle, a chemical composition of the skin may at be determined by using the spectral property in an intermediate step, wherein the estimated age of the skin may be determined by using the chemical composition. However, the estimated age of the skin may also be derivable directly from the spectral property without determining a full chemical composition of the skin before. Specifically, optically determining the estimated age of the skin may comprise considering at least one spectral property of the skin, specifically a spectral characteristic such as an absorption peak, referring to at least one material, specifically to water and/or fat, wherein the fat may also be denoted as lipid or adipose tissue. Generally, in the spectral range of 1.11 μm to 1.15 μm, in which a total absorption is generally dominated by water absorption, absorbance goes up with increasing age. In the spectral range of 1.17 μm to 1.21 μm, in which a total absorption is dominated by fat absorption, absorbance goes down with age. In the spectral range of 1.3 μm to 1.6 μm, in which a total absorption is dominated by water absorption, absorbance goes up with increasing age. In the spectral range of 1.68 μm to 1.79 μm, in which a total absorption is dominated by fat absorption, absorbance goes down with age. In the spectral range of 1.82 μm to 2 μm, in which a total absorption is dominated by water absorption, absorbance goes up with increasing age. For example based on these findings, the estimated age of the skin may be determined when evaluating an absorption spectrum of the skin as the skilled person will understand. The absorption spectrum may again for example be determined in at least one spectral measurement by using reflection spectroscopy as the skilled person will understand.

The method of optically determining an estimated age of a skin of a living being comprises:
i. using at least one sample reflection spectrum of at least a portion of the skin of the living being over a spectral measurement range, the spectral measurement range comprising at least a portion of the wavelength range of 1 μm to 2.5 μm; and
ii. determining the estimated age of the skin of the living being by applying, to the sample reflection spectrum, at least one trained trainable model,
   wherein the trainable model is trained on a training dataset comprising a plurality of labeled reference reflection spectra, each of the reference reflection spectra being acquired over a spectral range at least partially overlapping with the spectral measurement range of the sample reflection spectrum of step i.,
   wherein each of the reference reflection spectra is a reflection spectrum of at least one portion of a skin of a living test being having a known age,
   wherein the reference reflection spectra are at least partially labeled with at least the known age of the corresponding living test being.

The method steps may be performed in the given order. It shall be noted, however, that a different order may also be possible. The method may comprise further method steps which are not listed. Further, one or more of the method steps may be performed once or repeatedly. Further, two or more of the method steps may be performed simultaneously or in a timely overlapping fashion.

The at least one sample reflection spectrum, as an example, may be used in the form of a given data set, which may, as an example, be retrieved from at least one of a data storage device, a data transmission network or a measurement device for measuring the reflection spectrum. The method, thus, may be entirely be implemented as a computer-implemented method, by retrieving data such as data comprising the at least one sample reflection spectrum, and optionally by providing output data, the output data comprising at least one item of information on the estimated age of the skin of the living being.

Thus, the method may not comprise the step of actually measuring the sample reflection spectrum. The sample reflection spectrum may be provided in an electronic format and may be evaluated. Still, the method, as will be outlined in further detail below, may also further comprise providing the at least one sample reflection spectrum. The providing of the sample reflection spectrum may comprise at least one step of measuring the sample reflection spectrum.

The term “reflection” including any grammatical variations thereof as used herein is a broad term and is to be given its ordinary and customary meaning to a person of ordinary skill in the art and is not to be limited to a special or customized meaning. The term specifically may refer, without limitation, to a change in direction of a wave front of electromagnetic radiation, specifically infrared radiation, at an interface between two different optical media, wherein the wave front is returned into the optical medium from which it originated. The reflection may comprise specular reflection, also denoted as regular reflection. The reflection may specifically comprise diffuse reflection, wherein the incident electromagnetic radiation is back scattered at the interface into a plurality of different directions. Thus, the term “sample reflection spectrum” as used herein is a broad term and is to be given its ordinary and customary meaning to a person of ordinary skill in the art and is not to be limited to a special or customized meaning. The term specifically may refer, without limitation, to a spectrum determined by measuring at least one reflection of electromagnetic radiation, specifically infrared radiation, by at least one sample. The sample reflection spectrum may comprise a spectral distribution of a reflectivity of a sample. Further, the sample reflection spectrum may comprise a spectral distribution of a physical property determined by measuring a reflection of electromagnetic radiation by the sample. Specifically, the sample reflection spectrum may comprise a spectral distribution of an absorbance of the sample, wherein the absorbance may be determined by using a reflectivity of the sample, wherein the reflectivity may be determined by measuring a reflection of electromagnetic radiation by the sample. Thus, the sample reflection spectrum may specifically comprise at least one absorption spectrum determined by measuring at least one reflection of electromagnetic radiation by the sample. The sample may specifically be or may comprise at least a portion of a skin of a living being.

The term “portion of the skin” as used herein is a broad term and is to be given its ordinary and customary meaning to a person of ordinary skill in the art and is not to be limited to a special or customized meaning. The term specifically may refer, without limitation, to a part and/or a fraction of the skin. Thus, the portion of the skin may specifically refer to a spatially limited area of the skin on which at least one spectral measurement was performed for providing the sample reflection spectrum. The area may be closed such as a closed circle. The area may comprise a plurality of different and/or spread spots, e.g. closed circles, on which the spectral measurement was performed. Further, the portion of the skin may comprise at least one layer of the skin, e.g. the epidermis, depending on a penetration depth of electromagnetic radiation during the spectral measurement.

The term “spectral range” as used herein is a broad term and is to be given its ordinary and customary meaning to a person of ordinary skill in the art and is not to be limited to a special or customized meaning. The term specifically may refer, without limitation, to at least one wavelength interval, specifically from a minimum wavelength to a maximum wavelength, and/or to at least one individual wavelength. Thus, as an example, the spectral range may comprise a combination of different wavelength intervals and/or individual wavelengths. The term “spectral measurement range” as used herein is a broad term and is to be given its ordinary and customary meaning to a person of ordinary skill in the art and is not to be limited to a special or customized meaning. The term specifically may refer, without limitation, to a spectral range used for at least one spectral measurement for providing the sample reflection spectrum.

As said, the estimated age of the skin of the living being is determined by applying, to the sample reflection spectrum, at least one trained trainable model. The term “trainable model” as used herein is a broad term and is to be given its ordinary and customary meaning to a person of ordinary skill in the art and is not to be limited to a special or customized meaning. The term specifically may refer, without limitation, to a mathematical model for data interpretation, e.g. data classification, wherein the trainable model is trainable on at least one training dataset. In the trainable model, one or more parameters may still be variable, and the setting of the parameters is up to a training process. The trainable model may provide for a model of an environment, and the model may be adapted for reacting to stimuli from the environment in an adequate fashion and for adjusting the model in accordance with observed deviations such that, in a subsequent run, the model reacts to stimuli in a more adequate fashion. The trainable model may be trained on at least one training data set and may be configured for predicting at least one target variable for at least one input variable, such that, as an example, the at least one input variable forms a stimulus, and the output target variable forms the response of the trainable model. A plurality of trainable models is generally known to the skilled person and will be outlined in further detail below.

In a trained state, i.e. after full or partial training, the trainable model is also referred to as a “trained trainable model” or simply as a “trained model”. In the trained model, the parameters of the model may have been set, in order to reflect an appropriate reaction of the model to one or more stimuli, wherein the setting of the parameters and/or the set values of the parameters may be the result of the training process. Thus, the term “trained model” as used herein is a broad term and is to be given its ordinary and customary meaning to a person of ordinary skill in the art and is not to be limited to a special or customized meaning. The term specifically may refer, without limitation, to a trainable model, such as a mathematical model, which was trained on at least one training data set and which is configured for predicting at least one target variable for at least one input variable.

The term “training” including any grammatical variation thereof as used herein is a broad term and is to be given its ordinary and customary meaning to a person of ordinary skill in the art and is not to be limited to a special or customized meaning. The term specifically may refer, without limitation, to a process of building the at least one model, specifically by determining one or more parameters of the model, such as weights of the model and/or regression parameters.

The training may comprise at least one optimization and/or tuning process, wherein a parameter combination is determined.

As an example, the trainable model may be trained on a plurality of reflection spectra referring to a plurality of living beings having at least partially different known ages. In such case, the trainable model may be trained on how a reflection spectrum is composed for a selected known age, such that the trainable model can associate a new unknown reflection spectrum to an age on this basis. The trainable model may be retrained and/or updated based on at least one additional training dataset. The trained model may be trained by using at least one of machine learning, deep learning, neural networks, or other forms of artificial intelligence. As said, the trainable model is trained on a training dataset. The term “training dataset” as used herein is a broad term and is to be given its ordinary and customary meaning to a person of ordinary skill in the art and is not to be limited to a special or customized meaning. The term specifically may refer, without limitation, to data used or usable for training the trainable model. The training dataset may be known or predetermined. Specifically, the training dataset may be or may comprise historical experimental data such as previously experimentally determined data. Additionally or alternatively, the training dataset may comprise theoretical data such as theoretically calculated data, specifically simulated data.

As said, the training dataset comprises a plurality of labeled reference reflection spectra. The term “labeling” including any grammatical variation thereof as used herein is a broad term and is to be given its ordinary and customary meaning to a person of ordinary skill in the art and is not to be limited to a special or customized meaning. The term specifically may refer, without limitation, to providing at least one entity with at least one item of information on at least one property of the entity. The at least one item of information on at least one property of the entity is also referred to as the “label”. The at least one property of the entity, as an example, may be or may comprise at least one of a recognition sign, an identification sign and an information sign, which may all also be referred to as label. The label may comprise at least one item of information on at least one property of the labeled entity, wherein the item of information may specifically identify the labeled entity. The label may specifically be or may comprise a digital label.

The term “reference” as used herein is a broad term and is to be given its ordinary and customary meaning to a person of ordinary skill in the art and is not to be limited to a special or customized meaning. The term specifically may refer, without limitation, to an entity which is suitable for comparison purposes, specifically with respect to measured data. Thus, the reference may specifically comprise at least one predetermined or known property.

Consequently, the term “labeled reference reflection spectrum” as used herein is a broad term and is to be given its ordinary and customary meaning to a person of ordinary skill in the art and is not to be limited to a special or customized meaning. The term specifically may refer, without limitation, to a reflection spectrum relating to a reference, wherein the reflection spectrum is labeled with at least on item of information on the reference. Thus, specifically, the labeled reference reflection spectrum may be a reflection spectrum of at least one portion of a skin of a test person or living test being having a known age, wherein the label of the labeled reference reflection spectrum contains at least one item of information on the known age of the living test being.

As said, each of the reference reflection spectra is acquired over a spectral range at least partially overlapping with the spectral measurement range of step i. Each of the reference reflection spectra is a reflection spectrum of at least a portion of a skin of a living test being having a known age. The reference reflection spectra are at least partially labeled with at least the known age of the corresponding living test being. Thus, for at least a part of the reference reflection spectra, labels are provided which contain at least one item of information on the known age of the respective living test being.

The term “living test being” as used herein is a broad term and is to be given its ordinary and customary meaning to a person of ordinary skill in the art and is not to be limited to a special or customized meaning. The term specifically may refer, without limitation, to a living being used for training the trainable model. Thus, the living test being may specifically comprise at least one predetermined or known property. More specifically, as said, the living test being has a known age. The term “known age” as used herein is a broad term and is to be given its ordinary and customary meaning to a person of ordinary skill in the art and is not to be limited to a special or customized meaning. The term specifically may refer, without limitation, to a period of time, e.g. years, months or days, which has passed since a birth of the living being.

The spectral measurement range and the spectral ranges of the reference reflection spectra may be identical, or at least partially identical. In other words, the spectral measurement range may be identical with the spectral range of at least one of the reference reflection spectra. Thus, the spectral measurement range may extend over the same wavelengths and/or wavelength intervals as the spectral ranges of the reference reflection spectra.

The trained model or trained trainable model may be applied to the entire reflection spectrum or to just at least one part thereof and/or to at least one item of information derived from the reflection spectrum. Thus, the method may comprise deriving at least one item of information from the reflection spectrum and applying the trained trainable model to the at least one item of information, in order to determine the estimated age. As a simple example, one or more optical properties such as absorbance and/or reflectance at one or more pre-selected wavelengths may be derived from the reflection spectrum, and the trained trainable model may be applied to these optical properties in order to determine the estimated age. Thus, as an example, the trainable model may comprise a regression model deriving the estimated age from the one or more optical properties, and the training may imply determining the parameters such as the regression coefficients from the set of training data comprising the reference reflection spectra, e.g. by methods such as least squares regression and the like. The deriving of the one or more items of information may also comprise at least one preprocessing the reflection spectrum. Thus, the method may further comprise replacing the sample reflection spectrum by a preprocessed sample reflection spectrum being derived from the sample reflection spectrum. The term “preprocessing” including any grammatical variation thereof as used herein is a broad term and is to be given its ordinary and customary meaning to a person of ordinary skill in the art and is not to be limited to a special or customized meaning. The term specifically may refer, without limitation, to at least one of cleaning, filtering and transforming data. Cleaning data may for example comprise correcting incorrect and/or incomplete data, e.g. by using data wrangling tools. Filtering data may for example comprise removing irrelevant and/or useless data such as noise. Transforming data may specifically comprise performing analytical and/or statistical mathematical operations on the data, e.g. at least one of a derivation, an integration, a scaling and/or a normalization. The preprocessed sample reflection spectrum may specifically comprise at least one of: a first or higher order derivative of the sample reflection spectrum; a robust normal variate transform of the sample reflection spectrum; a filtered spectrum determined by filtering the sample reflection spectrum; a scaling of the sample reflection spectrum, wherein the scaling may comprise at least one of a unit scaling, a standard normal variate, i.e. a projection onto a unit sphere, and a range scaling.

Specifically, a plurality of preprocessing steps may be applied to the sample reflection spectrum. The preprocessing steps may specifically be adapted to the specific sample reflection spectrum. As an example, the sample reflection spectrum may be normalized by using a robust normal variate transform and a 50% percentile as described in Guo, Qian, Wen Wu, and D. L. Massart. “The robust normal variate transform for pattern recognition with near-infrared data.” Analytica chimica acta 382.1-2 (1999): 87-103. Subsequently, as an example, a second order derivative may be formed by using a Norris derivative filter as described in Hopkins, David W. “What is a Norris derivative?.” NIR news 12.3 (2001): 3-5. Subsequently, as an example, a unit scaling may be used for transforming features into a unit sphere, wherein specifically a Euclidian norm may be used. Subsequently, as an example, a partial least square regression may be applied as described in Abdi, Hervé. “Partial least square regression (PLS regression).” Encyclopedia for research methods for the social sciences 6.4 (2003): 792-795. However, as indicated, additional and/or alternative preprocessing steps may also be feasible.

As outlined above, generally, trainable models are known to the skilled person in the field of artificial intelligence and machine learning. However, for the purpose indicated herein, various trainable models are favorable. Thus, specifically, the trainable model may comprise at least one trainable model selected from the group consisting of: a principle component analysis model; a regression model, specifically a partial least square regression model; a principle component regression model; a lasso regression model; a nearest neighbor model; an artificial neural network, specifically an artificial neural network selected from the group consisting of a deep neural network, a convolutional neural network, a recurrent neural network, a long-short-term neural network; a support vector machine model; a decision tree classifier model; a decision tree classificatory, specifically at least one of a Random Forrest Classifier and a Boosted Decision Tree Classifier. It shall be noted that combinations of trainable models are also feasible, such as combinations comprising at least one trainable model selected from the list as indicated above. Further, hybrid models are also feasible.

Specifically, the trainable model may comprise at least one principal component regression model. The term “principal component regression model” as used herein is a broad term and is to be given its ordinary and customary meaning to a person of ordinary skill in the art and is not to be limited to a special or customized meaning. The term specifically may refer, without limitation, to a hybrid model combining at least one principle component analysis model and at least one regression model. Specifically, the principal component regression model may use a principal component analysis for determining at least one regression coefficient. More specifically, only a principal components subset may be used for a regression. Thus, the principal component regression model may facilitate a dimension reduction by decreasing an effective number of parameters characterizing the underlying model, which may specifically be useful in settings comprising high-dimensional covariates.

The portion of the skin of the living being and the portions of the skin of the living test beings may be portions in the same region of the body. The portion of the skin of the living being and the portions of the skin of the living test beings may be portions selected from the group consisting of: a skin portion at the temple of the living being or the living test beings, respectively; a skin portion at the forehead of the living being or the living test beings, respectively; a skin portion at the face of the living being or the living test beings, respectively; a skin portion at the neck of the living being or the living test beings, respectively; a skin portion at the forearm of the living being or the living test beings, respectively; a skin portion of the living being or the living test beings being at least one of essentially uncovered by hair, not strongly covered by hair and hairless, at least up to a tolerance of at most 20% of the skin portion being covered by hair, specifically of at most 10%, more specifically of at most 5%.

The method may further comprise at least one training step for training the trainable model for use in step ii. With respect to the term “training” reference may be made to the definitions and options given above. The training step may comprise providing the labeled reference reflection spectra as defined in step ii. The labeled reference reflection spectra, as outlined above, each comprise a reflection spectrum of at least one portion of a skin of a living test being having a known age, and, at least partially, are labeled with at least the known age of the corresponding living test being. The training step may also comprise acquiring the reference reflection spectra and, at least partially, labeling the reference reflection spectra. Thus, the training step may comprise
providing a plurality of test beings, the test beings having known ages,
acquiring the reference reflection spectra of said test beings, and
at least partially labeling the reference reflection spectra with the respective known ages.

Thereby, a training data set may be assembled, comprising the labeled reference reflection spectra. With said training data set, the trainable model may be trained, thereby, as an example, adjusting and/or optimizing parameters of the trainable model such that, when the trained trainable model is applied to a sample reflection spectrum of a living being having an unknown age, the age of the living being may be estimated.

The specific kind of training may depend on the at least one trainable model applied to the sample reflection spectrum. Thus, as outlined above, in case a regression model is used, training methods such as least squares regression may be used. Other training approaches may apply in case of an artificial neural network and so forth. The method specifically may comprise using at least one of a supervised and a semi-supervised learning architecture, specifically a deep learning architecture. The term “deep learning” as used herein is a broad term and is to be given its ordinary and customary meaning to a person of ordinary skill in the art and is not to be limited to a special or customized meaning. The term specifically may refer, without limitation, to a method of using artificial intelligence (AI) for automatic model building. Deep learning may specifically comprise at least one neural network, wherein the neural network may comprise at least one input layer, one output layer and at least one hidden layer between the input layer and the output layer.

The term “supervised learning” as used herein is a broad term and is to be given its ordinary and customary meaning to a person of ordinary skill in the art and is not to be limited to a special or customized meaning. The term specifically may refer, without limitation, to a deep learning method using a completely labeled training dataset. The term “semi-supervised learning” as used herein is a broad term and is to be given its ordinary and customary meaning to a person of ordinary skill in the art and is not to be limited to a special or customized meaning. The term specifically may refer, without limitation, to a learning method using a partially labeled training dataset, so that a part of the training dataset may have to be assigned independently based on a built model.

The method, as outlined above, comprises using the at least one sample reflection spectrum. The sample reflection spectrum, as an example, may be given in the form of a data set, such as an electronic data set. The method may further comprise providing the at least one sample reflection spectrum of the at least one portion of the skin of the living being over a spectral measurement range, the spectral measurement range comprising the at least one portion of the wavelength range of 1 μm to 2.5 μm. The term “providing” as used herein is a broad term and is to be given its ordinary and customary meaning to a person of ordinary skill in the art and is not to be limited to a special or customized meaning. The term specifically may refer, without limitation, to at least one of generating, retrieving and selecting at least one item, such as at least one item of information, e.g. a set of data comprising the at least one sample reflection spectrum. The providing, thus, may also optionally comprise the actual spectroscopic measurements for generating the sample reflection spectrum.

In a further aspect of the present invention, a computer-implemented method of providing at least one recommendation to a human being is disclosed. The method comprises:
I. determining the estimated age of the skin of the human being by using the method according to any one of the embodiments disclosed above or below in further detail referring to a method of optically determining an estimated age of a skin of a living being; and
II. automatically selecting the at least one recommendation for the human being based on the estimated age of the human being.

The method steps may be performed in the given order. It shall be noted, however, that a different order may also be possible. The method may comprise further method steps which are not listed. Further, one or more of the method steps may be performed once or repeatedly. Further, two or more of the method steps may be performed simultaneously or in a timely overlapping fashion.

The term “automatic” including any grammatical variation thereof as used herein is a broad term and is to be given its ordinary and customary meaning to a person of ordinary skill in the art and is not to be limited to a special or customized meaning. The term specifically may refer, without limitation, to a process which is performed at least partially without the necessity of human interaction, such as at least partially by a machine. Specifically, the process may be performed at least partially by means of at least one of a controller, a computer, a computer network and a machine, in particular without manual action and/or interaction with a user.

The term “recommendation” as used herein is a broad term and is to be given its ordinary and customary meaning to a person of ordinary skill in the art and is not to be limited to a special or customized meaning. The term specifically may refer, without limitation, to at least one item of information regarding at least one suggested action. Thus, the recommendation may comprise at least one item of information regarding at least one of a suggestion for taking at least one action, a suggestion for refraining from taking at least one action and a suggestion for modifying at least one action. The at least one recommendation specifically may refer to at least one of: a cosmetic treatment recommendation, specifically a treatment with at least one of a moisturizer and a skin cream with oil; a nutritional recommendation; a recommendation regarding at least one of a use of drugs and a use of medication; a recommendation regarding an exposition to at least one of sunlight and ultraviolet radiation; a recommendation regarding a use of sun screen; a recommendation to seek for medical consultation; a recommendation regarding sleeping habits; a recommendation regarding exercise; a recommendation regarding exposure to stress; a recommendation regarding the need for at least one of rest and vacation. In step II, the recommendation may be selected on the basis of a discrepancy between the estimated age of the human being and an actual age of the human being. Specifically, the higher the estimated age of the human being is than the actual age of the human being, the more extensive the recommendation may be.

The automatically selecting of the at least one recommendation may be performed by using at least one relation relating at least one of the estimated age and a discrepancy between the estimated age of the human being and an actual age of the human being to the at least one recommendation. The term “relation” including any grammatical variation thereof as used herein is a broad term and is to be given its ordinary and customary meaning to a person of ordinary skill in the art and is not to be limited to a special or customized meaning. The term specifically may refer, without limitation, to an allocation or an assignment of at least one first entity to at least one second entity. As indicated, the relation may relate the estimated age to at least one recommendation. Additionally or alternatively, the relation may relate a discrepancy between the estimated age of the human being and an actual age of the human being to the at least one recommendation. As an example, a case, wherein the estimated age is higher than the actual age of the human being, may be related to a cosmetic treatment recommendation such as a treatment with a moisturizer. The relation may comprise at least one of a lookup-table; an algorithm and a model.

In a further aspect of the present invention, a computer-implemented training method of training the trainable model for use in step ii. of the method according to any one of the embodiments disclosed above or below in further detail referring to a method of optically determining an estimated age of a skin of a living being is disclosed. The method comprises training the trainable model on the training dataset as defined in step ii. The method may comprise further method steps which are not listed. Further, one or more of the method steps may be performed once or repeatedly. Further, two or more of the method steps may be performed simultaneously or in a timely overlapping fashion. For possible definitions and/or options of the training, reference may be made to the embodiments of the computer-implemented method of optically determining an estimated age of a skin of a living being as described above.

Referring to the computer-implemented aspects of the invention, one or more of the method steps or even all of the method steps of the methods according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing samples and/or certain aspects of performing actual measurements.

In a further aspect of the present invention, a system for optically determining an estimated age of a skin of a living being is disclosed. The system is configured for performing the method according to any one of the embodiments disclosed above or below in further detail referring to a method of optically determining an estimated age of a skin of a living being. The system comprises:
A. at least one infrared spectrometer configured for acquiring the at least one sample reflection spectrum; and
B. at least one processing unit configured for performing the determining of the estimated age of the skin of the living being according to step ii.

The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components or parts forming a whole. Specifically, the components may interact with each other in order to fulfill at least one common function. The at least two components may be handled independently or may be coupled or connectable.

The term “infrared spectrometer” as used herein is a broad term and is to be given its ordinary and customary meaning to a person of ordinary skill in the art and is not to be limited to a special or customized meaning. The term specifically may refer, without limitation, to an arbitrary apparatus configured for determining spectral information by recording at least one measured value for at least one signal intensity related to at least one corresponding signal wavelength of infrared radiation and by evaluating at least one detector signal which relates to the signal intensity. Specifically, the spectrometer may be, may comprise or may be part of at least one miniaturized apparatus. More specifically, the spectrometer may be, may comprise or may be part of at least one handheld apparatus and/or at least one wearable device. The spectrometer may comprise at least one housing. The housing may be configured for protecting and/or shielding parts inside the housing against environmental influences such as mechanical influences or electromagnetic influences. The infrared spectrometer may comprise at least one infrared spectrometer selected from the group consisting of: a benchtop NIR spectrometer; a handheld NIR spectrometer; a spectroscopy module being part of at least one wearable device, specifically at least one wearable device selected from the group consisting of a smartwatch and a smartphone.

The term “processing unit” as used herein is a broad term and is to be given its ordinary and customary meaning to a person of ordinary skill in the art and is not to be limited to a special or customized meaning. The term specifically may refer, without limitation, to an arbitrary device adapted to perform the named operations, preferably by using at least one data processing device and, more preferably, by using at least one processor and/or at least one application-specific integrated circuit. As an example, the processing unit may comprise at least one data processing device having a software code stored thereon comprising a number of computer commands. The processing unit may provide one or more hardware elements for performing one or more of the named operations and/or may provide one or more processors with software running thereon for performing one or more of the named operations. As an example, the processing unit may comprise one or more programmable devices such as one or more computers, application-specific integrated circuits (ASICs), Digital Signal Processors (DSPs), or Field Programmable Gate Arrays (FPGAs) which are configured to perform the determining of the estimated age of the skin of the living being. Additionally or alternatively, however, the processing unit may also fully or partially be embodied by hardware. The processing unit may further be configured for controlling the infrared spectrometer or parts thereof. The processing unit may specifically be configured for performing at least one measurement cycle. The information as determined by the processing unit may be provided to at least one of a further apparatus and/or to a user, specifically in at least one of an electronic, visual, acoustic, or tactile fashion. The information as determined by the processing unit may be stored in a memory storage and/or in a separate storage device and/or may be passed on via at least one interface, such as a wireless interface and/or a wire-bound interface.

The processing unit may at least partially be cloud-based. The term “cloud-based” as used herein is a broad term and is to be given its ordinary and customary meaning to a person of ordinary skill in the art and is not to be limited to a special or customized meaning. The term specifically may refer, without limitation, to an outsourcing of the processing unit or of parts of the processing unit to interconnected external devices, specifically computers or computer networks having larger computing power and/or data storage volume. The external devices may be arbitrarily spatially distributed. The external devices may vary over time, specifically on demand. The external devices may be interconnected by using the internet. The external devices may each comprise at least one interface, such as a wireless interface and/or a wire-bound interface, specifically at least one communication interface.

In a further aspect of the present invention, a computer program is disclosed. The computer program comprises instructions which, when the program is executed by the system according to any one of the embodiments disclosed above or below in further detail referring to a system, cause the system to perform at least one of: the method according to any one of the embodiments disclosed above or below in further detail referring to a method; the training method according to any one of the embodiments disclosed above or below in further detail referring to a training method.

In a further aspect of the present invention, a computer-readable storage medium is disclosed. The computer-readable storage medium comprises instructions which, when the program is executed by the system according to any one of the embodiments disclosed above or below in further detail referring to a system, cause the system to perform at least one of: the method according to any one of the embodiments disclosed above or below in further detail referring to a method; the training method according to any one of the embodiments disclosed above or below in further detail referring to a training method. The computer-readable storage medium may refer to non-transitory data storage means, such as a hardware storage medium having stored thereon computer-executable instructions. The computer-readable storage medium specifically may be or may comprise at least one storage medium such as a random-access memory (RAM) and/or a read-only memory (ROM).

The methods and devices according to the present invention may provide a large number of advantages over known methods and systems. In particular, they can provide information about the skin on a molecular level and may be robust against external interferences. Thus, the obtained results may be precise and reliable. Particularly, the methods and systems according to the present invention can convey information about deeper skin layers beyond the stratum corneum by using a non-destructive measurement method. The measurement can be performed with any spectroscopic device capable of measuring in the wavelength range of 1 μm to 2.5 μm and is, thus, widely applicable. In result, the methods and systems according to the present invention may be well suited for defining meaningful quantitative parameters for skin quality that can be the basis of particular steps to influence the skin in certain ways. They can specifically give feedback to a user such as specific and customized suggestions for a healthier living or an application of certain cosmetic products.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically are used only once when introducing the respective feature or element. In most cases, when referring to the respective feature or element, the expressions “at least one” or “one or more” are not repeated, notwithstanding the fact that the respective feature or element may be present once or more than once.

Further, as used herein, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features.

Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Summarizing and without excluding further possible embodiments, the following embodiments may be envisaged:

Embodiment 1: A computer-implemented method of optically determining an estimated age of a skin of a living being, specifically of a human being, the method comprising:
i. using at least one sample reflection spectrum of at least one portion of the skin of the living being over a spectral measurement range, the spectral measurement range comprising at least one portion of the wavelength range of 1 μm to 2.5 μm; and
ii. determining the estimated age of the skin of the living being by applying, to the sample reflection spectrum, at least one trained trainable model,
   wherein the trainable model is trained on a training dataset comprising a plurality of labeled reference reflection spectra, each of the reference reflection spectra being acquired over a spectral range at least partially overlapping with the spectral measurement range of the sample reflection spectrum of step i.,
   wherein each of the reference reflection spectra is a reflection spectrum of at least one portion of a skin of a living test being having a known age,
   wherein the reference reflection spectra are at least partially labeled with at least the known age of the corresponding living test being.

Embodiment 2: The method according to the preceding embodiment, wherein the spectral measurement range and the spectral ranges of the reference reflection spectra are identical.

Embodiment 3: The method according to any one of the preceding embodiments, further comprising replacing the sample reflection spectrum by a preprocessed sample reflection spectrum being derived from the sample reflection spectrum, the preprocessed sample reflection spectrum specifically comprising at least one of: a first or higher order derivative of the sample reflection spectrum; a robust normal variate transform of the sample reflection spectrum; a filtered spectrum determined by filtering the sample reflection spectrum; a scaling of the sample reflection spectrum, wherein the scaling comprises at least one of a unit scaling, a standard normal variate and a range scaling.

Embodiment 4: The method according to any one of the preceding embodiments, wherein the trainable model is selected from the group consisting of: a principle component analysis model; a regression model, specifically a partial least square regression model; a principle component regression model; a lasso regression model; a nearest neighbor model; an artificial neural network, specifically an artificial neural network selected from the group consisting of a deep neural network, a convolutional neural network, a recurrent neural network, a long-short-term neural network; a support vector machine model; a decision tree classifier model; a decision tree classificatory, specifically at least one of a Random Forrest Classifier and a Boosted Decision Tree Classifier.

Embodiment 5: The method according to any one of the preceding embodiments, wherein the portion of the skin of the living being and the portions of the skin of the living test beings are portions in the same region of the body.

Embodiment 6: The method according to any one of the preceding embodiments, wherein the portion of the skin of the living being and the portions of the skin of the living test beings are portions, selected from the group consisting of: a skin portion at the temple of the living being or the living test beings, respectively; a skin portion at the forehead of the living being or the living test beings, respectively; a skin portion at the face of the living being or the living test beings, respectively; a skin portion at the neck of the living being or the living test beings, respectively; a skin portion at the forearm of the living being or the living test beings, respectively; a skin portion not strongly covered by hair and/or a hairless skin portion of the living being or the living test beings, respectively, at least up to a tolerance of at most 20% of the skin portion being covered by hair, specifically of at most 10%, more specifically of at most 5%.

Embodiment 7: The method according to any one of the preceding embodiments, further comprising providing the at least one sample reflection spectrum of the at least one portion of the skin of the living being over a spectral measurement range, the spectral measurement range comprising the at least one portion of the wavelength range of 1 μm to 2.5 μm.

Embodiment 8: The method according to any one of the preceding embodiments, the method further comprising at least one training step for training the trainable model for use in step ii., the training step comprising providing the labeled reference reflection spectra as defined in step ii., the method further comprising using at least one of a supervised and a semi-supervised deep learning architecture.

Embodiment 9: A computer-implemented method of providing at least one recommendation to a human being, the method comprising:
I. determining the estimated age of the skin of the human being by using the method according to any one of the preceding embodiments; and
II. automatically selecting the at least one recommendation for the human being based on the estimated age of the human being.

Embodiment 10: The method according to the preceding embodiment, wherein the at least one recommendation refers to at least one of: a cosmetic treatment recommendation, specifically a treatment with at least one of a moisturizer and a skin cream with oil; a nutritional recommendation; a recommendation regarding at least one of a use of drugs and a use of medication; a recommendation regarding an exposition to at least one of sunlight and ultraviolet radiation; a recommendation regarding a use of sun screen; a recommendation to seek for medical consultation; a recommendation regarding sleeping habits; a recommendation regarding exercise; a recommendation regarding exposure to stress; a recommendation regarding the need for at least one of rest and vacation.

Embodiment 11: The method according to any one of the two preceding embodiments, wherein, in step II, the recommendation is selected on the basis of a discrepancy between the estimated age of the human being and an actual age of the human being.

Embodiment 12: The method according to any one of the three preceding embodiments, wherein the automatically selecting of the at least one recommendation is performed by using at least one relation relating at least one of the estimated age and a discrepancy between the estimated age of the human being and an actual age of the human being to the at least one recommendation.

Embodiment 13: A computer-implemented training method of training the trainable model for use in step ii. of the method according to any one of the preceding embodiments referring to a method of optically determining an estimated age of a skin of a living being, the method comprising training the trainable model on the training dataset as defined in step ii.

Embodiment 14: A system for optically determining an estimated age of a skin of a living being, the system being configured for performing the method according to any one of the preceding embodiments referring to a method of optically determining an estimated age of a skin of a living being, the system comprising:
A. at least one infrared spectrometer configured for acquiring the at least one sample reflection spectrum; and
B. at least one processing unit configured for performing the determining of the estimated age of the skin of the living being according to step ii.

Embodiment 15: The system according to the preceding embodiment, wherein the infrared spectrometer comprises at least one infrared spectrometer selected from the group consisting of: a benchtop NIR spectrometer; a handheld NIR spectrometer; a spectroscopy module being part of at least one wearable device, specifically at least one wearable device selected from the group consisting of a smartwatch and a smartphone.

Embodiment 16: The system according to any one of the two preceding embodiments, wherein the processing unit is at least partially cloud-based.

Embodiment 17: A computer program comprising instructions which, when the program is executed by the system according to any one of the preceding embodiments referring to a system, cause the system to perform at least one of the method according to any one of the preceding embodiments referring to a method and the computer-implemented training method according to any one of the preceding embodiments referring to a computer-implemented training method

Embodiment 18: A computer-readable storage medium comprising instructions which, when the program is executed by the system according to any one of the preceding embodiments referring to a system, cause the system to perform at least one of the method according to any one of the preceding embodiments referring to a method and the training method according to any one of the preceding embodiments referring to a training method.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 schematically shows an exemplary embodiment of a system for optically determining an estimated age of a skin of a living being;

FIG. 2 shows a flow chart of an embodiment of a computer-implemented method of optically determining an estimated age of a skin of a living being;

FIGS. 3A-3E show exemplary sample reflection spectra of skins of living beings;

FIGS. 4A-4B show exemplary estimated ages determined by using an embodiment of a computer-implemented method of optically determining an estimated age of a skin of a living being;

FIG. 5 shows a flow chart of an embodiment of a computer-implemented method of providing at least one recommendation to a human being; and

FIG. 6 shows a flow chart of an embodiment of a computer-implemented training method of training a trainable model for use in a computer-implemented method of optically determining an estimated age of a skin of a living being.

### DETAILED DESCRIPTION OF THE EMBODIMENTS

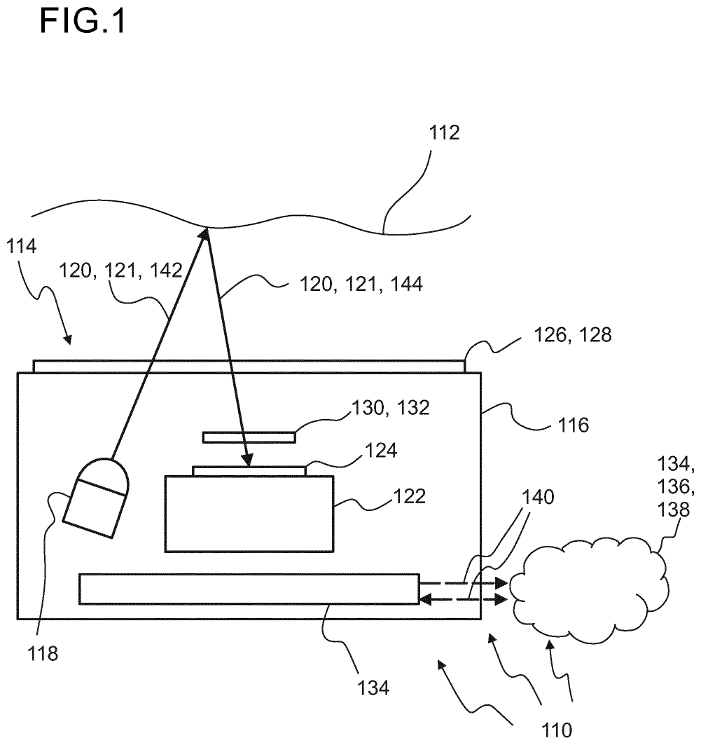

FIG. 1 schematically shows an exemplary embodiment of a system 110 for optically determining an estimated age of a skin 112 of a living being, specifically a human being. The system 110 is configured for performing a method of optically determining an estimated age of the skin 112 of a living being according to any one of the embodiments disclosed above or below in further detail referring to a method of optically determining an estimated age of the skin 112 of a living being. The skin 112 may comprise an arbitrary texture such as a curved texture. The system 110 comprises at least one infrared spectrometer 114 configured for acquiring at least one sample reflection spectrum. The infrared spectrometer 114 may comprise at least one infrared spectrometer 114 selected from the group consisting of: a benchtop NIR spectrometer; a handheld NIR spectrometer; a spectroscopy module being part of at least one wearable device, specifically at least one wearable device selected from the group consisting of a smartwatch and a smartphone.

As FIG. 1 shows, the system 110 may comprise at least one housing 116. The housing 116 may at least partially encompass components of the system 110, specifically the infrared spectrometer 114. In this fashion, the components of the infrared spectrometer 114 may be protected and access of external light may be impeded. However, further kinds of arrangements may also be conceivable. The infrared spectrometer 114 may comprise at least one radiation emitting element 118 configured for emitting optical radiation 120, specifically infrared radiation 121. The radiation emitting element 118 may be comprised by a semiconductor-based radiation source which may be selected from at least one of a light emitting diode or a laser, specifically a laser diode. However, further kinds of radiation emitting element 118 may also be feasible. The radiation emitting element 118 may be configured for emitting continuously or for generating modulated optical pulses. The infrared spectrometer 114 may further comprise at least one photosensitive detector 122 comprising at least one photosensitive region 124 designated for receiving the optical radiation 120. The photosensitive detector 122 may be configured for generating at least one detector signal depending on an illumination of the photosensitive region 124. The infrared spectrometer 114 may comprise at least one optical element 126, specifically at least one optical window 128. The infrared spectrometer 114 may comprise at least one spectral transfer element 130 configured for selecting at least one wavelength of the optical radiation 120. The spectral transfer element 130 may specifically comprise at least one optical filter 132. For further details regarding the infrared spectrometer 114, reference may for example be made to EP application number 20211174.6 filed on Dec. 2, 2020.

The system 110 further comprises at least one processing unit 134 configured for performing a determining of the estimated age of the skin 112 of the living being. The processing unit 134 may at least partially be cloud-based. In other words, the processing 134 unit may at least partially be distributed in a cloud 136 used for at least one of cloud computing and cloud storage. The cloud 136 may specifically comprise at least one external device 138 being located outside the housing 116, e.g. a computer or a computer network. The external device 138 may be connected to the infrared spectrometer 114, specifically to a portion of the processing unit 134 being located inside the housing 116, by at least one interface 140 for data transfer. The interface 140 may be wireless and/or wire-bound.

As FIG. 1 shows, the radiation emitting element 118 may emit optical radiation 120, specifically infrared radiation 121, as incident optical radiation 142 towards the skin 112 of the living being. The incident optical radiation 142 may pass the optical element 126, specifically the optical window 128, before reaching the skin 112. The skin 112 may at least partially reflect, specifically diffusely, the optical radiation 120 towards the photosensitive region 124 of the photosensitive detector 122 in form of reflected optical radiation 144. Further, the skin may at least partially absorb the incident optical radiation 142. The reflected optical radiation 144 may pass the optical element 126, specifically the optical window 128, before reaching the photosensitive region 124. The reflected optical radiation 140 may further pass the spectral transfer element 130, specifically the optical filter 132, before reaching the photosensitive region 124. The photosensitive detector 122 may generate at least one detector signal depending on a corresponding illumination of the photosensitive region 124. The photosensitive detector 122 may specifically transmit the detector signal towards the processing unit 134 for further processing. The processing unit 134 may distribute at least one item of information generated by using the detector signal within the cloud 136 for further processing and/or storage.

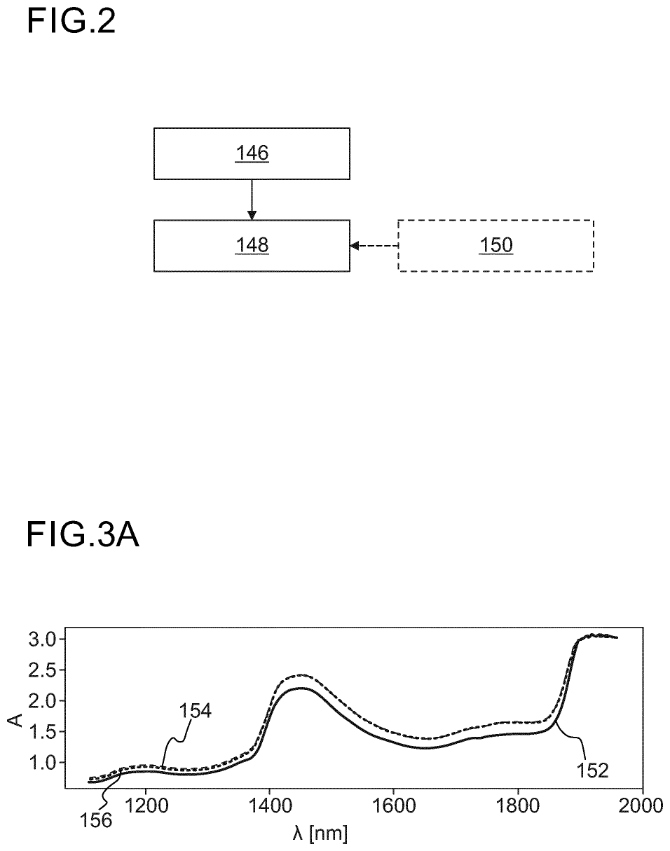

FIG. 2 shows a flow chart of an embodiment of a computer-implemented method of optically determining the estimated age of the skin 112 of the living being. The method comprises the following method steps:
i. (denoted with reference number 146) using at least one sample reflection spectrum of at least one portion of the skin 112 of the living being over a spectral measurement range, the spectral measurement range comprising at least one portion of the wavelength range of 1 μm to 2.5 μm; and
ii. (denoted with reference number 148) determining the estimated age of the skin 112 of the living being by applying, to the sample reflection spectrum, at least one trained trainable model,
   wherein the trainable model is trained on a training dataset comprising a plurality of labeled reference reflection spectra, each of the reference reflection spectra being acquired over a spectral range at least partially overlapping with the spectral measurement range of the sample reflection spectrum of step i.,
   wherein each of the reference reflection spectra is a reflection spectrum of at least one portion of a skin 112 of a living test being having a known age,
   wherein the reference reflection spectra are at least partially labeled with at least the known age of the corresponding living test being.

The method steps may be performed in the given order. It shall be noted, however, that a different order may also be possible. The method may comprise further method steps which are not listed. Further, one or more of the method steps may be performed once or repeatedly. Further, two or more of the method steps may be performed simultaneously or in a timely overlapping fashion.

The spectral measurement range and the spectral ranges of the reference reflection spectra may be identical. The method may further comprise replacing the sample reflection spectrum by a preprocessed sample reflection spectrum being derived from the sample reflection spectrum. The preprocessed sample reflection spectrum may specifically comprise at least one of: a first or higher order derivative of the sample reflection spectrum; a robust normal variate transform of the sample reflection spectrum; a filtered spectrum determined by filtering the sample reflection spectrum; a scaling of the sample reflection spectrum, wherein the scaling may comprise at least one of a unit scaling, a standard normal variate and a range scaling. The trainable model may be selected from the group consisting of: a principle component analysis model; a regression model, specifically a partial least square regression model; a principle component regression model; a lasso regression model and/or lasso regressor; a nearest neighbor model; an artificial neural network, specifically an artificial neural network selected from the group consisting of a deep neural network, a convolutional neural network, a recurrent neural network, a long-short-term neural network; a support vector machine model; a decision tree classifier model; a decision tree classificatory, specifically at least one of a Random Forrest Classifier and a Boosted Decision Tree Classifier. The portion of the skin 112 of the living being and the portions of the skin 112 of the living test being may be portions of the same region of the body. The portion of the skin 112 of the living being and the portions of the skin 112 of the living test beings may be portions, selected from the group consisting of: a skin portion at the temple of the living being or the living test beings, respectively; a skin portion at the forehead of the living being or the living test beings, respectively; a skin portion at the face of the living being or the living test beings, respectively; a skin portion at the neck of the living being or the living test beings, respectively; a skin portion at the forearm of the living being or the living test beings, respectively; a skin portion not strongly covered by hair and/or a hairless skin portion of the living being or the living test beings, respectively, such as a skin portion being covered by hair up to an area ratio hairy skin surface/total skin surface of no more than 20%, specifically no more than 10%, more specifically no more than 5%. The method may further comprise providing the at least one sample reflection spectrum of the at least one portion of the skin 112 of the living being over a spectral measurement range, the spectral measurement range comprising the at least one portion of the wavelength range of 1 μm to 2.5 μm.

The method may further comprise at least one training step 150 for training the trainable model for use in step ii. The training step 150 may comprise providing the labeled reference reflection spectra as defined in step ii. The method may further comprise using at least one of a supervised and a semi-supervised deep learning architecture. The training step 150 may also comprise acquiring the reference reflection spectra and, at least partially, labeling the reference reflection spectra. Thus, the training step 150 may comprise
providing a plurality of test beings, the test beings having known ages,
acquiring the reference reflection spectra of said test beings, and
at least partially labeling the reference reflection spectra with the respective known ages.

Thereby, a training data set may be assembled, comprising the labeled reference reflection spectra. With said training data set, the trainable model may be trained, thereby, as an example, adjusting and/or optimizing parameters of the trainable model such that, when the trained trainable model is applied to a sample reflection spectrum of a living being having an unknown age, the age of the living being may be estimated.

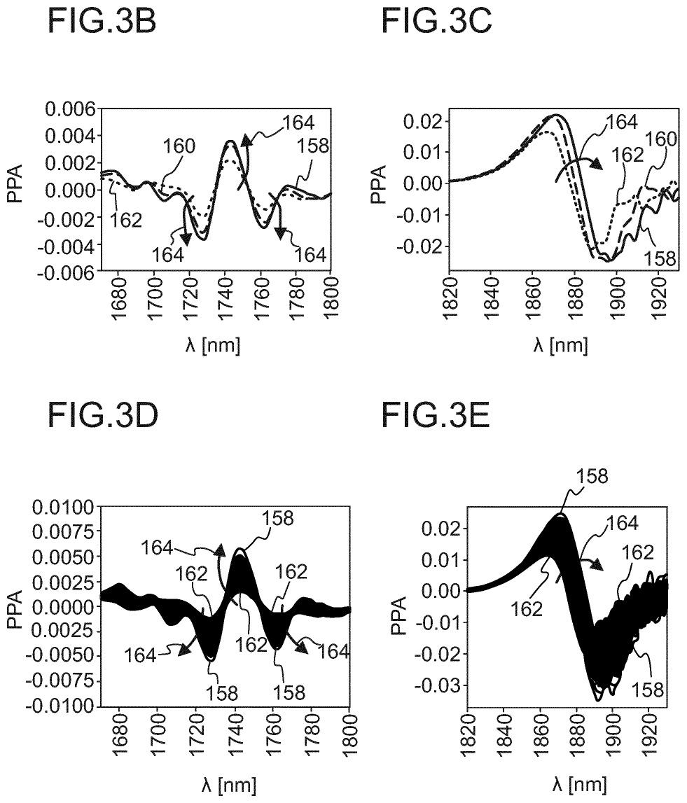

FIGS. 3A-3E show exemplary sample reflection spectra of the skins 112 of living beings, specifically of human beings. FIG. 3A shows measured absorbances of the skins 112 of a 8 year old human being, a 39 year old human being and a 59 year old human being. The absorbance was measured by using a FT-NIR Multi Purpose Analyzer (MPA) by Bruker Corp. in reflection mode and is given in arbitrary units in the Figures. The term “absorbance” is abbreviated by the Latin letter A and the term “wavelength” is denoted with the Greek letter λ in the Figures. The absorbance measured for the 8 year old human being is denoted with reference number 152 and is printed as continuous line. The absorbance measured for the 39 year old human being is denoted with reference number 154 and is printed as dashed line. The absorbance measured for the 59 year old human being is denoted with reference number 156 and is printed as dotted line. In particular, the absorbances measured for the 39 year old human being and the 59 year old human being lie close together in FIG. 3A. However, as will be shown below with respect to FIGS. 3B to 3E, through suitable preprocessing of the measured absorbances, several age-dependent features will become clearly visible.

FIGS. 3B and 3C show corresponding preprocessed absorbances for selected wavelengths in arbitrary units. The term “preprocessed absorbance” is abbreviated as PPA in the Figures. The preprocessed absorbance of the 8 year old human being is denoted by reference number 158 and is printed as continuous line. The preprocessed absorbance of the 39 year old human being is denoted by reference number 160 and is printed as dashed line. The preprocessed absorbance of the 59 year old human being is denoted by reference number 162 and is printed as dotted line. For obtaining the preprocessed absorbances PPA of FIGS. 3B and 3C, the measured absorbances A were normalized by using a robust normal variate transform and a 50% percentile as described in Guo, Qian, Wen Wu, and D. L. Massart. “The robust normal variate transform for pattern recognition with near-infrared data.” Analytica chimica acta 382.1-2 (1999): 87-103. Subsequently, second order derivatives were formed by using a Norris derivative filter as described in Hopkins, David W. “What is a Norris derivative?.” NIR news 12.3 (2001): 3-5. Subsequently, a unit scaling was used for transforming features into a unit sphere, wherein specifically a Euclidian norm may be used. Subsequently, a partial least square regression was applied as described in Abdi, Hervé. “Partial least square regression (PLS regression).” Encyclopedia for research methods for the social sciences 6.4 (2003): 792-795. FIGS. 3B and 3C illustrate that several features of the preprocessed absorbance distinguishably change with varying known age of the analyzed human being. As an example, in FIG. 3B, in the wavelength rage from 1720 nm to 1760 nm, maxima and minima of the preprocessed absorbance are more prominent for decreasing age. As a further example, in FIG. 3C, in the wavelength range from 1840 nm to 1900 nm, a maximum of the preprocessed absorbance shifts towards higher wavelengths A for decreasing age. The addressed progression from older age to younger age is indicated by arrows 164 in FIGS. 3B and 3C.

As FIGS. 3D and 3E show, the trends described above with respect to the preprocessed absorbances for an 8 year old human being, a 39 year old human being and a 59 human being are also evident for larger groups of human beings and are thus statistically reliable. For FIGS. 3D and 3E, 29 human beings having known ages ranging from 8 years to 59 years were analyzed in total. The term “known age”, also referred to as “actual age”, is abbreviated as KA in the Figures. Analogously to FIG. 3B, in FIG. 3D it is evident that, in the wavelength rage from 1720 nm to 1760 nm, the maxima and minima of the preprocessed absorbance are more prominent for decreasing age. Analogously to FIG. 3C, in FIG. 3E it is evident that, in the wavelength range 1840 nm to 1900 nm, the maximum of the preprocessed absorbance shifts towards higher wavelengths A for decreasing age. As before, the addressed progression from older age to younger age is indicated by arrows 164 in FIGS. 3D and 3E. Thus, for example based on these findings, it may be possible to train the trainable model used in step ii. of the method of optically determining the estimated age of the skin 112 of the living being and to further on assign an estimated age of the skin 112 to unknown samples.

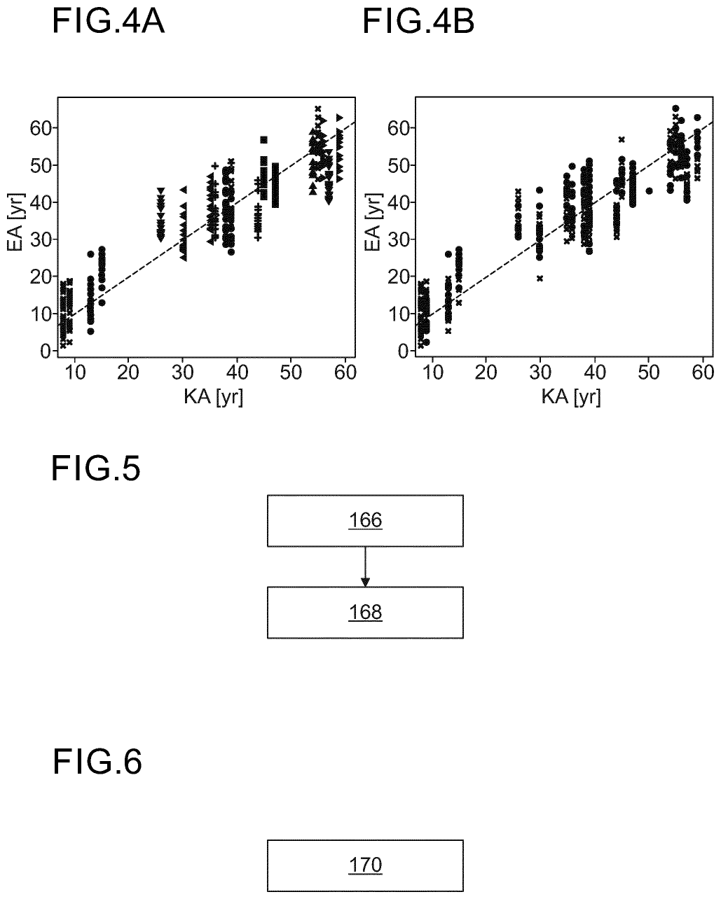

FIGS. 4A and 4B show exemplary estimated ages of the skin 112 determined by using an embodiment of a computer-implemented method of optically determining an estimated age of the skin 112 of a living being. The term “estimated age” is abbreviated as EA in the Figures. For each analyzed human being 20 independent measurements were conducted. The measured sample reflection spectra were subsequently preprocessed as described above, before the trained trainable model was applied to them for determining an estimated age of the skin 112. In FIG. 4A, different symbols such as circles or crosses are used for different human beings. In FIG. 4B, circles indicate that the skin 112 of a temple of a human being was analyzed, whereas crosses indicate that the skin 112 of a forehead of a human being was analyzed. For FIG. 4B, 10 measurements were conducted on the skin 112 of the temple and 10 measurements were conducted on the skin 112 of the forehead for each analyzed human being. FIGS. 4A and 4B reveal a clear correlation between a known age of the human being on the x-axis and an estimated age of the skin 112 of the human being on the y-axis. Generally, a deviation between a known age of a living being and an estimated age of a living being can be used for providing at least one recommendation.

FIG. 5 shows a flow chart of an embodiment of a computer-implemented method of providing at least one recommendation to a human being. The method comprises the following method steps:
I. (denoted with reference number 166) determining an estimated age of the skin 112 of the human being by using the method according to any one of the embodiments disclosed above or below in further detail referring to a method of optically determining an estimated age of the skin 112 of a living being; and
II. (denoted with reference number 168) automatically selecting the at least one recommendation for the human being based on the estimated age of the human being.

The method steps may be performed in the given order. It shall be noted, however, that a different order may also be possible. The method may comprise further method steps which are not listed. Further, one or more of the method steps may be performed once or repeatedly. Further, two or more of the method steps may be performed simultaneously or in a timely overlapping fashion.

The at least one recommendation may refer to at least one of: a cosmetic treatment recommendation, specifically a treatment with at least one of a moisturizer and a skin cream with oil; a nutritional recommendation; a recommendation regarding at least one of a use of drugs and a use of medication; a recommendation regarding an exposition to at least one of sunlight and ultraviolet radiation; a recommendation regarding a use of sun screen; a recommendation to seek for medical consultation; a recommendation regarding sleeping habits; a recommendation regarding exercise; a recommendation regarding exposure to stress; a recommendation regarding the need for at least one of rest and vacation. In step II, the recommendation may be selected on the basis of a discrepancy between the estimated age of the human being and an actual age of the human being. The automatically selecting of the at least one recommendation may be performed by using at least one relation relating at least one of the estimated age and a discrepancy between the estimated age of the human being and an actual age of the human being to the at least one recommendation. The relation may comprise at least one of a lookup-table; an algorithm and a model.

FIG. 6 shows a flow chart of an embodiment of a computer-implemented training method of training a trainable model for use in step ii. of the method of optically determining an estimated age of the skin 112 of a living being according to any one of the embodiments disclosed above or below in further detail referring to a method of optically determining an estimated age of the skin 112 of a living being. The training method (denoted with reference number 170) comprises training the trainable model on the training dataset as defined in step ii.

The method may comprise further method steps which are not listed. Further, one or more of the method steps may be performed once or repeatedly. Further, two or more of the method steps may be performed simultaneously or in a timely overlapping fashion.

Referring to the computer-implemented aspects of the invention, one or more of the method steps or even all of the method steps of the methods according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing samples and/or certain aspects of performing actual measurements.

### LIST OF REFERENCE NUMBERS

110 system for optically determining an estimated age
112 skin of a living being
114 infrared spectrometer
116 housing
118 radiation emitting element
120 optical radiation
121 infrared radiation
122 photosensitive detector
124 photosensitive region
126 optical element
128 optical window
130 spectral transfer element
132 optical filter
134 processing unit
136 cloud
138 external device
140 interface
142 incident optical radiation
144 reflected optical radiation
146 method step i.
148 method step ii.
150 training step
152 measured absorbance for a 8 year old human being
154 measured absorbance for a 39 year old human being
156 measured absorbance for a 59 year old human being
158 preprocessed absorbance for a 8 year old human being
160 preprocessed absorbance for a 39 year old human being
162 preprocessed absorbance for a 59 year old human being
164 arrow indicating progression from older age to younger age
166 method step I.
168 method step II.
170 training method
A absorbance
λ wavelength
PPA preprocessed absorbance
KA known age
EA estimated age

1. A computer-implemented method of optically determining an estimated age of a skin of a living being, the method comprising:
i. using at least one sample reflection spectrum of at least one portion of the skin of the living being over a spectral measurement range, the spectral measurement range comprising at least one portion of the wavelength range of from 1 μm to 2.5 μm; and
ii. determining the estimated age of the skin of the living being by applying, to the sample reflection spectrum, at least one trained trainable model,
wherein the trainable model is trained on a training dataset comprising a plurality of labeled reference reflection spectra, each of the reference reflection spectra being acquired over a spectral range at least partially overlapping with the spectral measurement range of the sample reflection spectrum of step i.,
wherein each of the reference reflection spectra is a reflection spectrum of at least one portion of a skin of a living test being having a known age,
wherein the reference reflection spectra are at least partially labeled with at least the known age of the corresponding living test being,
wherein the trainable model is selected from the group consisting of a principle component analysis model; a regression model; a nearest neighbor model; an artificial neural network; a support vector machine model; a decision tree classifier model; and a decision tree classificatory,
the method further comprising at least one training step for training the trainable model for use in step ii., the training step comprising providing the labeled reference reflection spectra as defined in step ii., the method further comprising using at least one of a supervised and a semi-supervised learning architecture.

2. The method according to claim 1, wherein the spectral measurement range and the spectral ranges of the reference reflection spectra are identical.

3. The method according to claim 1, further comprising replacing the sample reflection spectrum by a preprocessed sample reflection spectrum being derived from the sample reflection spectrum, the preprocessed sample reflection spectrum specifically comprising at least one of a first or higher order derivative of the sample reflection spectrum; a robust normal variate transform of the sample reflection spectrum; a filtered spectrum determined by filtering the sample reflection spectrum; or a scaling of the sample reflection spectrum, wherein the scaling comprises at least one of a unit scaling, a standard normal variate or a range scaling.

4. The method according to claim 1, wherein the portion of the skin of the living being and the portions of the skin of the living test beings are portions in the same region of the body.

5. The method according to claim 1, wherein the portion of the skin of the living being and the portions of the skin of the living test beings are portions, selected from the group consisting of a skin portion at the temple of the living being or the living test beings, respectively; a skin portion at the forehead of the living being or the living test beings, respectively; a skin portion at the face of the living being or the living test beings, respectively; a skin portion at the neck of the living being or the living test beings, respectively; a skin portion at the forearm of the living being or the living test beings, respectively; and a skin portion essentially not covered by hair and/or a hairless skin portion of the living being or the living test beings, respectively, with at least up to a tolerance of at most 20% of the skin portion being covered by hair.

6. The method according to claim 1, further comprising providing the at least one sample reflection spectrum of the at least one portion of the skin of the living being over a spectral measurement range, the spectral measurement range comprising the at least one portion of the wavelength range of from 1 μm to 2.5 μm.

7. A computer-implemented method of providing at least one recommendation to a human being, the method comprising:
I. determining the estimated age of the skin of the human being by using the method according to claim 1; and
II. automatically selecting the at least one recommendation for the human being based on the estimated age of the human being.

8. The method according to claim 7, wherein the at least one recommendation refers to at least one of a cosmetic treatment recommendation; a nutritional recommendation; a recommendation regarding at least one of a use of drugs and a use of medication; a recommendation regarding an exposition to at least one of sunlight and ultraviolet radiation; a recommendation regarding a use of sun screen; a recommendation to seek for medical consultation; a recommendation regarding sleeping habits; a recommendation regarding exercise; a recommendation regarding exposure to stress; or a recommendation regarding the need for at least one of rest and vacation.

9. The method according to claim 7, wherein, in step II, the recommendation is selected on the basis of a discrepancy between the estimated age of the human being and an actual age of the human being.

10. The method according to claim 7, wherein the automatically selecting of the at least one recommendation is performed by using at least one relation relating at least one of the estimated age and a discrepancy between the estimated age of the human being and an actual age of the human being to the at least one recommendation.

11. A computer-implemented training method of training the trainable model for use in step ii. of the method according to claim 1, the method comprising training the trainable model on the training dataset as defined in step ii.

12. A system for optically determining an estimated age of a skin of a living being, the system being configured for performing the method according to claim 1, the system comprising:
A. at least one infrared spectrometer configured for acquiring the at least one sample reflection spectrum; and
B. at least one processing unit configured for performing the determining of the estimated age of the skin of the living being according to step ii.

13. The system according to claim 12, wherein the infrared spectrometer comprises at least one infrared spectrometer selected from the group consisting of a benchtop NIR spectrometer; a handheld NIR spectrometer; and a spectroscopy module being part of at least one wearable device.

14. The method according to claim 1, wherein the living being is a human being.

15. The method according to claim 1, wherein step ii. the regression model is selected from the group consisting of a partial least square regression model; a principle component regression model; and a lasso regression model.

16. The method according to claim 1, wherein step ii. the artificial neural network is selected from the group consisting of a deep neural network, a convolutional neural network, a recurrent neural network, and a long-short-term neural network.

17. The method according to claim 1, wherein step ii. the decision tree classificatory is at least one of a Random Forrest Classifier or a Boosted Decision Tree Classifier.

18. The method according to claim 8, wherein the cosmetic treatment recommendation is a treatment with at least one of a moisturizer and a skin cream with oil.

19. The system according to claim 13, wherein the at least one wearable device is selected from the group consisting of a smartwatch and a smartphone.
